(19) United States
(12) Patent Application Publication
(10) Pub. No.: US 2013/0145526 A1
(43) Pub. Date: Jun. 13, 2013

(54) FACE MASK WITH LENS RETAINING ASSEMBLY

(71) Applicant: LESTER V. BROERSMA, San Diego, CA (US)

(72) Inventor: LESTER V. BROERSMA, San Diego, CA (US)

(21) Appl. No.: 13/316,942

(22) Filed: Dec. 12, 2011

(51) Int. Cl. A61F 9/04

(52) U.S. Cl. 2/427

(57) ABSTRACT

A protective face mask with a mask body and a removable lens. A lens retaining assembly connects the lens to the mask body. Typically, the lens retaining assembly includes a retaining plug and a locking strap to selectively capture the lens. The locking strap flexes between a locked and open position.

### TECHNICAL FIELD

This invention relates generally to face masks; in particular, a lens retaining assembly for a face mask.

### BACKGROUND

Face masks are used to protect the user's face and eyes in a wide variety of sports. In the sport of paintball, for example, players are required to wear masks to prevent risk of injury if a paintball would impact a player's face or eyes. Several paintball masks have been developed that utilize a removable lens. These removable lenses are installed and captured on either the inside or outside of the mask.

Several lens-capturing systems have been developed over the years. One of the problems with these systems is the use of several loose components, which may become misplaced or lost during lens replacement. Others are difficult to operate, or questionable as to their effectiveness. Therefore, there is a need for a face mask with a new lens capturing system that overcomes these deficiencies.

### SUMMARY

According to one aspect, the present invention provides a protective face mask with a lens retaining assembly. The face mask includes a mask body and a lens. The mask body includes a lens opening for receiving a lens. A first mask connection opening and a second mask connection opening are defined in the mask body. The lens is dimensioned to be received in the lens opening. The lens includes a first lens connection opening and a second lens connection alignable with the first mask connection opening and the second mask connection opening, respectively, of the mask body. In this embodiment, the lens retaining assembly includes retaining plugs and locking straps for each side of the mask body and lens. The retaining plugs have a shank extending from a head. The shank is dimensioned to extend through one of the mask connection openings and lens connection openings when the lens retaining assembly is connected with the lens and mask body. The head has a greater dimension than the mask connection openings to prevent the head from passing therethrough. The locking straps include a first segment spaced apart from a second segment. The segments are configured to be attached to the retaining plugs. When the segments are connected, a portion of the lens is retained on the retaining plug between the segments.

In some embodiments, the shank of the retaining plugs are dimensioned to extend longitudinally through the mask connection opening and the lens connection opening when the lens retaining assembly is connected with the lens and mask body. Typically, the locking straps are flexible and formed from a resilient material. Depending on the circumstances, embodiments are contemplated in which the segments of the locking strap are separated by a portion of the lens when the first locking strap is connected to the first retaining plug. In some cases, the first segment of the locking straps have a hole dimensioned to receive at least a portion of the shank of the retaining plugs. For example, the hole in the first segment could be configured to deform to receive the shank of the retaining plug. In some embodiments, the retaining plugs include a retaining ring with an annular wall that has a greater dimension than the hole in the first segment so that the hole in the first segment of the first locking strap must stretch to pass over the retaining ring.

Embodiments are contemplated in which the retaining plugs include a passageway dimensioned to receive at least a portion of the second segment of the locking straps. For example, the passageway may extend along a transverse axis. In some cases, the second segment could include one or more detents dimensioned to be received by one or more notches defined in the passageway when the second segment is received in the passageway.

According to another aspect, the present invention provides a protective face mask with a lens retaining assembly. The face mask includes a mask body and a lens. The mask body includes a lens opening for receiving a lens. A first mask connection opening and a second mask connection opening are defined in the mask body. The lens is dimensioned to be received in the lens opening. The lens includes a first lens connection opening and a second lens connection alignable with the first mask connection opening and the second mask connection opening, respectively, of the mask body. In this embodiment, the lens retaining assembly includes retaining plugs for each side of the mask body and lens. The lens retaining assembly includes means for retaining at least a portion of the lens on the retaining plugs. Typically, when the lens is connected with the mask body, the means is flexed about its longitudinal axis.

According to yet another aspect, the present invention provides a protective face mask with a lens retaining assembly. The face mask includes a mask body and a lens. The mask body includes a lens opening for receiving a lens. A first mask connection opening and a second mask connection opening are defined in the mask body. The lens is dimensioned to be received in the lens opening. The lens includes a first lens connection opening and a second lens connection alignable with the first mask connection opening and the second mask connection opening, respectively, of the mask body. In this embodiment, the lens retaining assembly includes retaining plugs and locking straps for each side of the mask body and lens. The retaining plugs extend through one of the mask connection openings and lens connection openings when the lens retaining assembly is connected with the lens and mask body. The locking straps include a first segment and a second segment. The second segment is moveable between an open position and a closed position. In the closed position, the second segment is connected to the retaining plug such that the locking strap captures at least a portion of the lens. In the open position, the second segment is disconnected from the retaining plug to release the portion of the lens. Typically, the locking strap is flexed about its longitudinal axis when the second segment is connected to the retaining plug.

According to another aspect, the present invention provides a method for connecting a lens to a mask. The method includes the step of providing a mask body including a lens opening for receiving a lens. The mask body defines at least one mask connection opening. A lens dimensioned to be received in the lens opening is provided, such that the lens defines at least one lens connection opening. The mask connection opening is aligned with the lens connection opening. The method also includes the step of inserting a retaining plug through the mask connection opening and the lens connection opening. A first portion of a locking strap is connected to the retaining plug on a first side of the lens. A second portion of the locking strap is connected to the retaining plug on a second side of the lens. Typically, at least a portion of the lens is retained on the retaining plug between the first portion and the second portion of the locking strap.

Additional features and advantages of the invention will become apparent to those skilled in the art upon consideration of the following detailed description of the illustrated embodiment exemplifying the best mode of carrying out the invention as presently perceived. It is intended that all such additional features and advantages be included within this description and be within the scope of the invention.

### BRIEF DESCRIPTION OF THE DRAWINGS

The present disclosure will be described hereafter with reference to the attached drawings which are given as non-limiting examples only, in which:

FIG. 1 is a top view of an example paintball mask with a removable lens assembly that can be placed on a user's head for protection;

FIG. 2 is a right side view of an example mask assembly with the ends of the lens assembly extending into the mask body;

FIG. 3 is a partial view of an example lens attachment assembly with an interior mounting configuration according to an embodiment of the present invention;

FIG. 4 is a top view of an example retaining plug according to an embodiment of the present invention;

FIG. 5 is a cross-sectional view of the example retaining plug along line 5-5 of FIG. 4;

FIG. 6 is an end view of the example retaining plug shown in FIG. 4;

FIG. 7 is a cross-sectional view of the retaining plug along line 7-7 of FIG. 6;

FIG. 8 is a front view of an example locking strap according an embodiment of the present invention;

FIG. 9 is a cross-sectional view of the locking strap shown in FIG. 8 along line 9-9;

FIG. 10 is an exploded view of the lens attachment assembly with a portion of a mask and lens;

FIG. 11 is a cross-sectional view of the lens attachment assembly along line 11-11 of FIG. 3;

FIG. 12 is a side view of the lens attachment assembly shown in FIG. 3;

FIG. 13 is a cross-sectional view of the lens attachment assembly along line 13-13 of FIG. 12;

FIG. 14 is a perspective view of an example locking strap according to another embodiment;

FIG. 15 is a top view of an example retaining plug according to another embodiment of the present invention along line 15-15 of FIG. 16;

FIG. 16 is a side view of the example retaining plug shown in FIG. 15 with the example locking strap shown in FIG. 14;

FIG. 17 is an end view of the example retaining plug and locking strap shown in FIG. 16;

FIG. 18 is a cross-sectional view of the example retaining plug and locking strap shown in FIG. 16 in an assembled state along line 18-18 of FIG. 19;

FIG. 19 is a cross-sectional view of the example retaining plug and locking strap shown in FIG. 17 in an assembled state along line 19-19 of FIG. 18;

FIG. 20 is a cross-sectional view of the example retaining plug and locking strap shown in FIG. 17 in an assembled state along line 20-20 of FIG. 19; and

FIGS. 21A-21H show various positions and shapes for the retaining plug and locking strap accordingly to various embodiments of the present invention.

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

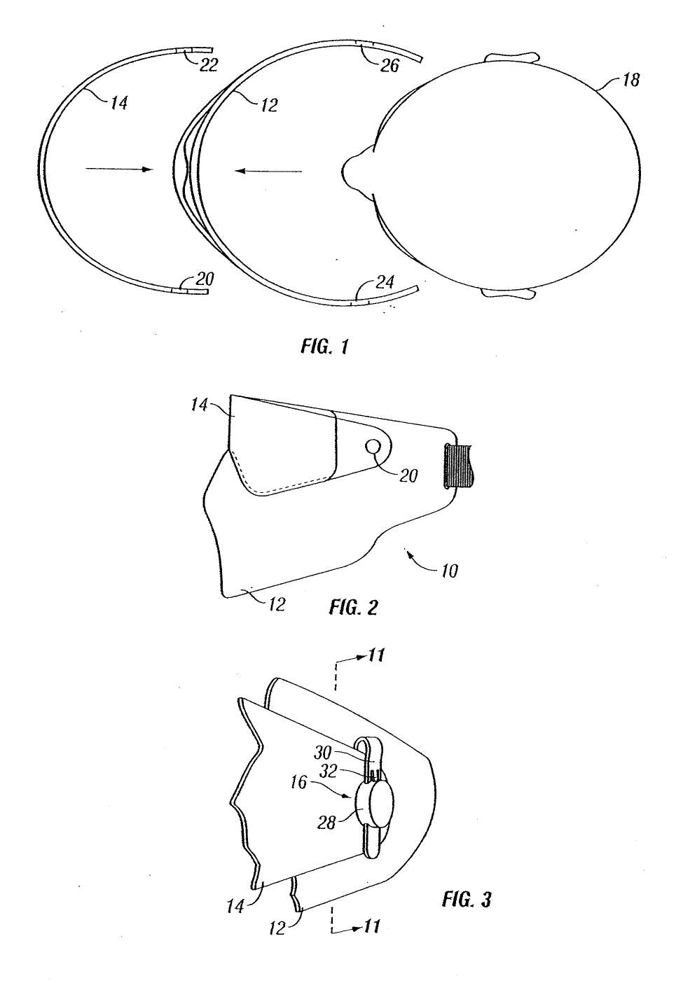

Referring to the drawings, FIGS. 1 and 2 show a protective mask 10 with a mask body 12, a lens 14, and a lens retaining assembly 16 (FIGS. 3-20). In the example shown in FIG. 1, a top exploded view of the protective mask 10 is shown with a head 18 of a user, along with the mask body 12 and lens 14. The mask body 12 and lens 14 may be coupled together with the lens retaining assembly 16 such that the lens 14 may be attached and disconnected from the mask body 12. (See FIGS. 3-20.)

In the example shown, the lens 14 includes a first lens opening 20 and a second lens opening 22 that may be aligned with a first mask connection opening 24 and a second mask connection opening 26, respectively. With these openings 20, 22, 24, 26 aligned, as explained in more detail below, the lens retaining assembly 16 may be used to couple the lens 14 together with the mask body 12. The terms “couple,” “coupled,” “connect,” and “connected” are intended broadly to encompass both direct and indirect connections. Thus, the lens 14 is said to be connected together with the mask body 12 when they are directly connected (i.e., by direct contact, such as interference or frictional fit), as well as when the lens retaining assembly 16 indirectly couples the lens 14 and mask body 12.

FIG. 2 shows a left side view of the example protective mask 10 with the first lens opening 20 aligned with the first mask connection opening 24. Although the example shown has the ends of the lens 14 inserted into the mask body 12, embodiments are also contemplated in which the lens 14 could be connected on the outside surface of the mask body 12. Typically, the lens 12 is formed from a clear or transparent material that can provide protection to the user's eyes, such as polycarbonate. The mask body is typically formed from a hard plastic, nylon or other materials that could provide protection.

FIG. 3 shows an embodiment of the lens retaining assembly 16 for connecting the lens 14 and mask body 12. In this embodiment, the lens retaining assembly 16 includes a first connection point on one side of the lens 14 and a second connection point on the other side of the lens 14 that traps the lens 14 therebetween. In the embodiment shown, the lens retaining assembly 16 includes a retaining plug 28 and a locking strap 30. As shown, the locking strap 30 has two connection points with the retaining plug 28 on each side of the lens 14 to trap the lens 14 therebetween. One of the connection points can be seen in FIG. 3. As shown, the locking strap 30 includes a segment that passes through a passageway of the retaining plug 28. The locking strap 30 includes a stop 32 that limits further insertion of the locking strap into the passageway of the retaining plug 28, which sets the position of the locking strap 30 that is retained on the retaining plug 28. Although not shown in FIG. 3, the locking strap 30 has another connection point on the other side of the lens 14, which is discussed below.

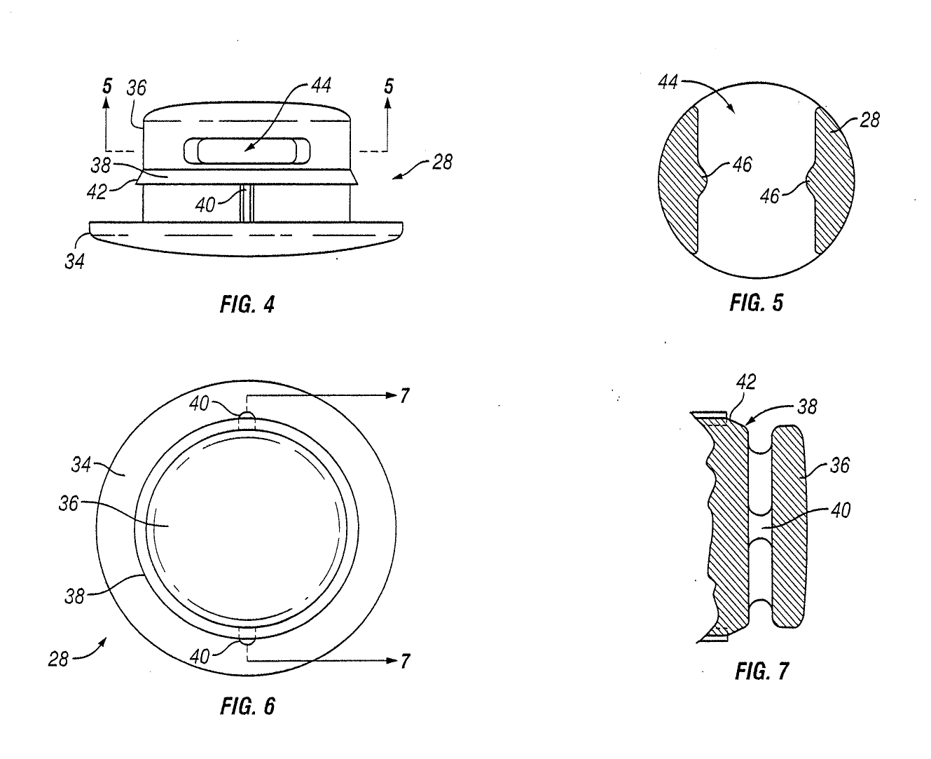

FIG. 4 is a top view of an example retaining plug 28 according to one embodiment. In the embodiment shown, the retaining plug 28 includes a head 34 that is dimensioned larger than the corresponding mask connection opening 24, 26 so that the retaining plug 28 does not pass therethrough. A shank 36 extends from the head of the retaining plug 28. In the embodiment shown, a retaining ring 38 is defined on the shank 36 as an annular wall to retain a portion of the locking strap 30, as discussed below. A fin 40 is defined between the head 34 and retaining ring 38 on the shank 36 that corresponds with a notch in the locking strap 30 as discussed below. Accordingly, the distance between the retaining ring 38 and the head 34 is dimensioned to secure a portion of the locking strap 30 therebetween. In the example shown, the retaining ring 38 includes a tapered portion 42 that allows a portion of the locking strap 30 to pass thereover. A passageway 44 is defined in the shank 36 along a transverse axis. The passageway 44 is dimensioned to receive a portion of the locking strap 30, as discussed below. In the example shown, the end of the shank 36 is chamfered to allow easy insertion into the mask connection openings 24, 26. The retaining plug 28 could be formed from hard plastic, metal, wood or other suitable rigid material.

FIG. 5 is a cross-sectional view of the retaining plug 28 along line 5-5 of FIG. 4. This view shows an embodiment in which the passageway 44 includes detents 46 for receiving notches in the locking strap 30. This limits movement of the locking strap to retain its position and provides feedback to the user that feels the interaction between the detents and notches.

FIG. 6 shows an end view of the retaining plug 28. In this example, the retaining plug 28 has fins 40 spaced approximately 180 degrees apart on the shank 36. Embodiments are contemplated in which a single fin or more than one fin could be arranged on the retaining plug 28 corresponding to slots in the locking strap 30. The interaction between the fins 40 and notches reduces rotation or twisting of the retaining plug 28 with respect to the locking strap 30.

FIG. 7 is a cross-sectional view of the retaining plug 28 along line 7-7 of FIG. 6. In this view, the annular wall of the retaining ring 38 can be seen. As mentioned before, the retaining ring 38 maintains the position of a portion of the locking strap 30.

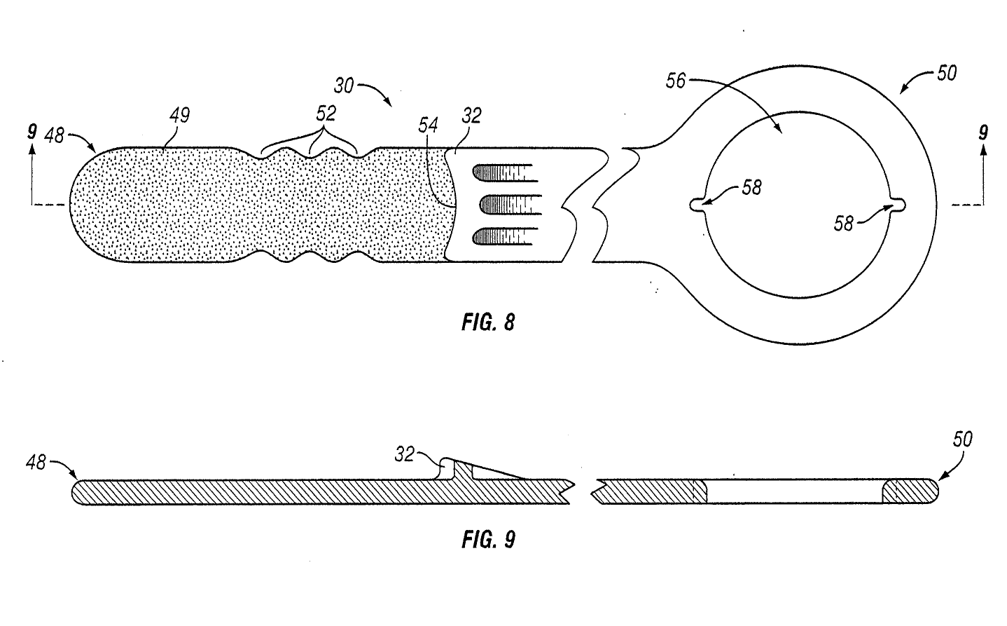

FIG. 8 shows a front view of the locking strap 30 according to one embodiment. In the example shown, the locking strap 30 includes a first end 48 and a second end 50. The first end 48 includes a first segment 49 that is received by the passageway 44 and the retaining plug 28. As shown in this example, the first segment 49 includes one or more notches 52 that are arranged to be received by the detent 46 inside the passageway 44. In some embodiments, the first segment 49 may include a textured surface that would allow the user to more easily grip and place the first segment 49 into the passageway of the retaining plug 28. The stop 32 includes an arcuate portion 54 that corresponds with the shape of the retaining plug 28 adjacent the passageway 44.

The second end 50 of the locking strap 30 defines an opening 56 that is dimensioned to receive the shank 36 of the retaining plug 28. In one embodiment, the locking strap 30 is formed from a flexible or resilient material, such as urethane, and the opening 56 may stretch or be deformed to extend over the retaining ring 38. In this example, the perimeter of the opening 56 includes notches 58 that are positioned to be received by the fins 40 on the shank 36 of the retaining plug 28. FIG. 9 shows a cross-sectional view of the locking strap 30 along line 9-9 of FIG. 8.

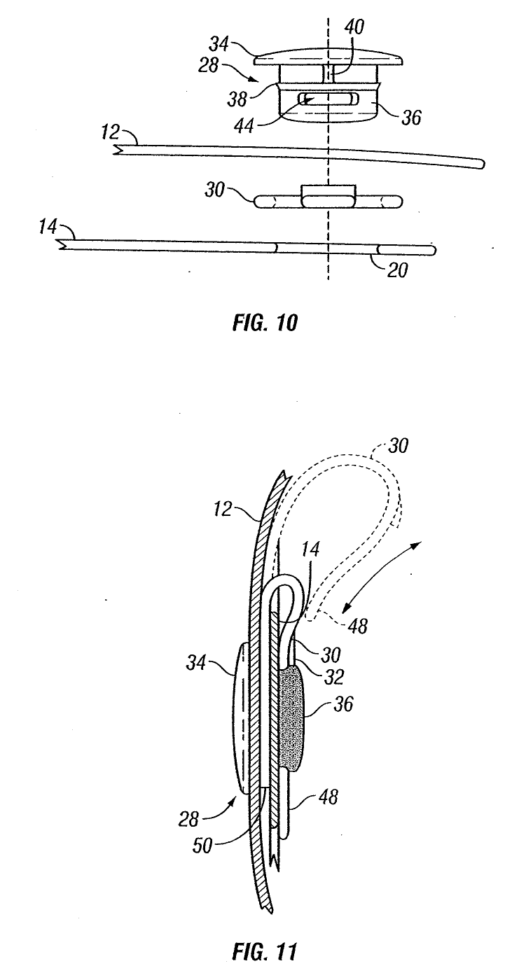

FIG. 10 shows an exploded view of the lens retaining assembly 16 according to an embodiment. In this example, a retaining plug 28 is configured with the head 34 to be received on the outside of the mask body 12. The shank 36 of the retaining plug 28 is received by the lens opening 20 as shown. In this example, the retaining ring 38 of the retaining plug 28 is configured to be received by the locking strap 30 such that the second end 50 of the locking strap 30 is retained between the mask body 12 and the lens 14.

FIG. 11 shows a side cross-sectional view of the lens retaining assembly 16 along line 11-11 of FIG. 3, according to the embodiment shown in FIG. 10. In this example, the shank 36 is received by the openings in the mask body 12 and lens 14. As shown, the second end 50 of the locking strap 30 is received on the shank 36 between the mask body 12 and the lens 14. For example, the user may insert the shank 36 into one of the openings 24, 26 in the mask body 12. The user could then insert the shank 36 through the opening 56 in the second end 50 of the locking strap. The shank 36 could then be inserted into the corresponding openings 20, 22 of the lens. The first end 48 of the locking strap is free to be inserted into the passageway 44 defined in the shank 36 of the retaining plug 28. This captures the lens 14 between the first end 48 and second end 50 of the locking strap 30. As the user inserts the first end 48 of the locking strap 30 into the passageway 44, the user receives feedback from feeling the interaction of the tapered portion 42 and detents 46 until the stop 32 prevents further insertion into the passageway 44. If the user desires to detach that portion of the lens 14 from the lens retaining assembly 16, the first end 48 of the locking strap 30 may be removed from the passageway 44 and the lens 14 removed from the shank 36 of the retaining plug 28. Shown in phantom on FIG. 11 is the first end 48 of the locking strap 30 flexing to be inserted into the passageway 44.

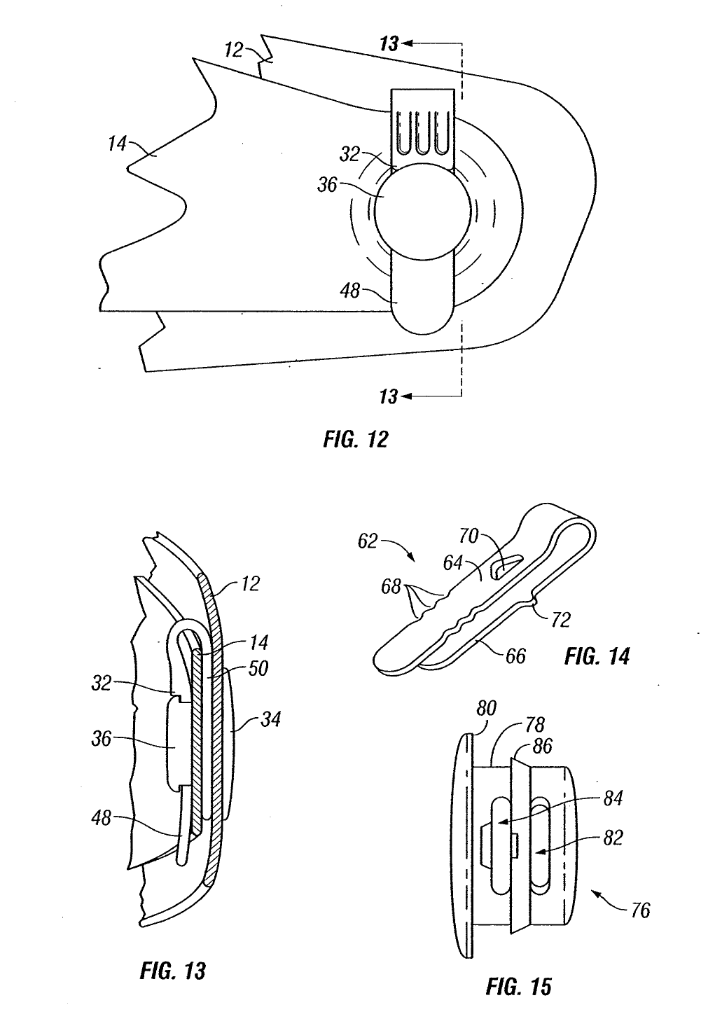

FIG. 12 is a side view of the mask 10 interior. As shown, the locking strap 30 has a second end 50 (obscured from the lens 14) that is received on the shank 36 between the lens 14 and the mask body 12. The first end 48 of the locking strap 30 has been inserted through the passageway 44 until further movement is prevented by the stop 32. In the example shown, the end of the stop 32 has an arcuate shape to conform with the cylindrical shape of the shank 36 in this embodiment.

FIG. 13 is a cross-sectional view of the lens mask 10 along line 13-13 of FIG. 12. In this example, the lens 14 is captured between the first end 48 and second end 50 of the locking strap 30.

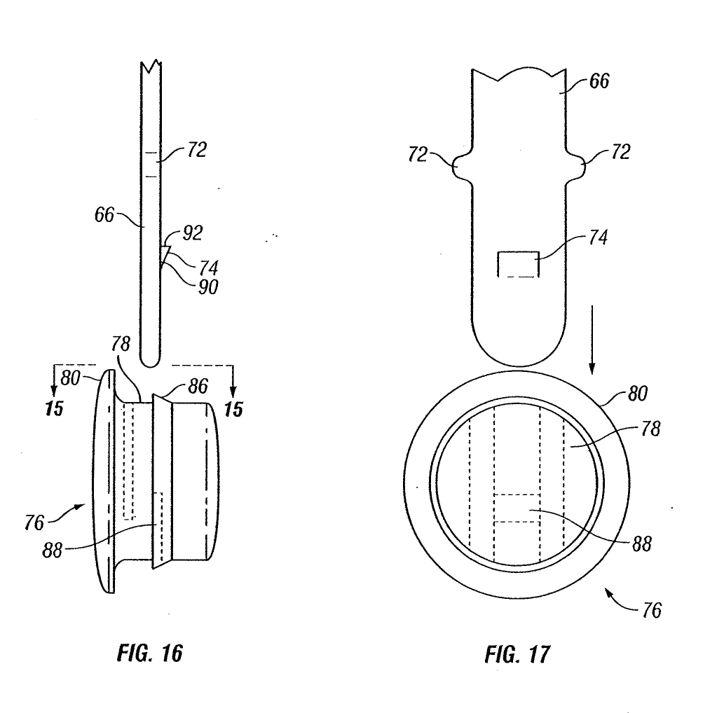

FIGS. 14-20 show a lens retaining assembly 60 according to another embodiment. In this embodiment, the locking strap 62 includes a first segment 64 and a second segment 66. Although the first segment 64 is similar to the first segment 49 described above, and includes notches 68 and a stop 70, the retaining plug in this embodiment has more than one passageway. The second segment 66 is dimensioned to be received in one of the passageways. The second segment 66 includes a stop 72 and a one-way lock 74 (FIGS. 16-17) to prevent removal of the second segment 66 from the retaining plug 76. This reduces the possibility that the locking strap 62 and the retaining plug 76 will become separated.

Referring to FIG. 15, in this embodiment, the retaining plug 76 has a shank 78 extending from a head 80. A first passageway 82 and a second passageway 84 extend transversely through the shank 78 of the retaining plug 76. As shown, a retaining ring 86 is disposed between passageways 82, 84. The first segment 64 is dimensioned to be received in the first passageway 82 and the second segment 66 is dimensioned to be received in the second passageway 84.

FIGS. 16 and 17 show a side and end view, respectively, of the second segment 66 prior to insertion into the second passageway 84 of the retaining plug 76. During insertion, the one-way lock 74 deforms to be received within the second passageway 84 until reaching a slot 88 in the second passageway 84. Upon reaching the slot 88, the one-way lock 74 “springs” back resiliently to block removal. In the embodiment shown, the one-way lock 74 includes a tapered portion 90 and a blocking wall 92. The tapered portion 90 allows insertion into the second passageway 84. The blocking wall 92 provides an interference fit with the retaining plug 76 after the tapered portion 90 “springs” back to shape upon reaching the slot 88.

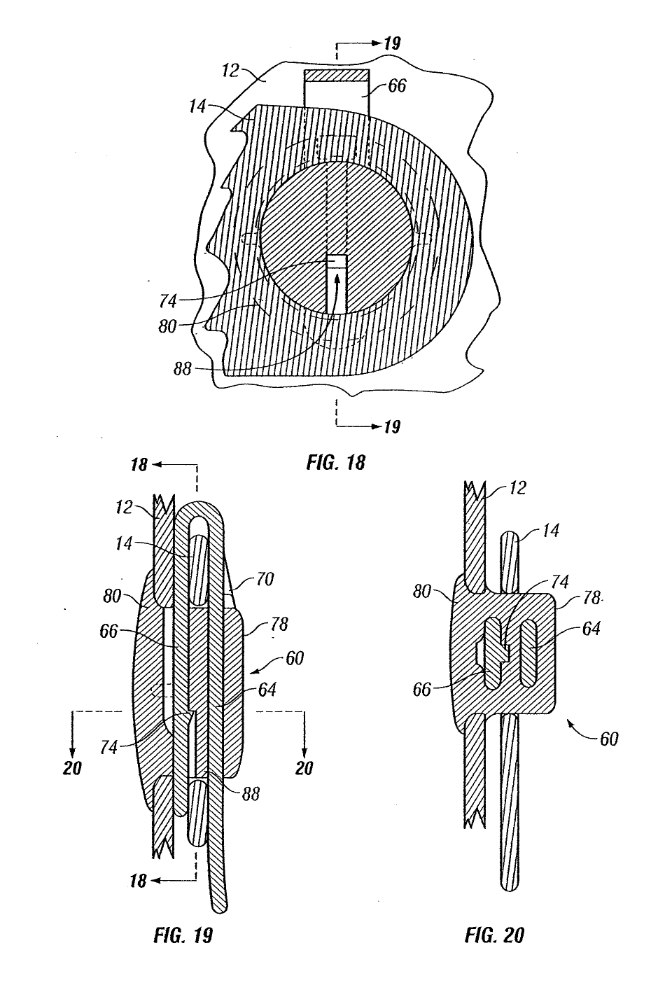

FIG. 18 is a cross-sectional view of the lens retaining assembly 60 from inside the mask 10 along line 18-18 of FIG. 19. As shown, the second segment 66 of the locking strap 62 has been received by the second passageway 84 of the retaining plug 60. As discussed above, the second passageway 84 includes a slot 88 that is dimensioned to receive the one-way lock 74. The blocking wall 92 creates an interference connection with the retaining plug 76

FIG. 19 shows a side cross-sectional view of the lens retaining assembly 60 along line 19-19 of FIG. 18. In this example, the one-way lock 74 can be seen in the slot 88 to prevent removal of the second segment 66. The first segment 64 of the locking strap 62 is flexed to capture the lens 14 between the first segment 64 and second segment 66. FIG. 20 shows a top cross-sectional view along line 20-20 of FIG. 19.

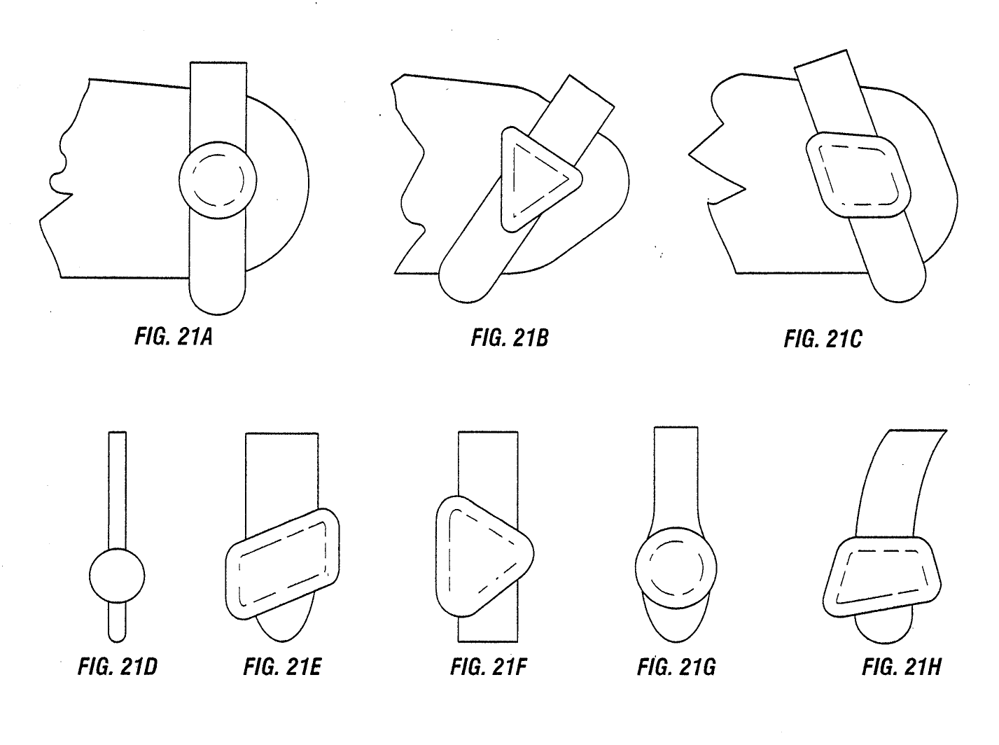

FIGS. 21a-21h show examples of various shapes that the head of the retaining plug could have. As shown, the head could be circular, triangular, rectangular, a parallel gram, or any other polygon shape or other shape. Likewise, the locking strap could have various shapes depending on the circumstances.

Although the present disclosure has been described with reference to particular means, materials, and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A protective face mask with lens retaining assembly comprising:
a mask body including a lens opening for receiving a lens, wherein the mask body defines a first mask connection opening and a second mask connection opening;
a lens dimensioned to be received in the lens opening, wherein the lens includes a first lens connection opening and a second lens connection alignable with the first mask connection opening and the second mask connection opening, respectively, of the mask body;
a lens retaining assembly comprising:
a first retaining plug having a shank extending from a head, wherein the shank is dimensioned to extend through the first mask connection opening and the first lens connection opening when the lens retaining assembly is connected with the lens and mask body, wherein the head has a greater dimension than the first mask connection opening to prevent the head from passing therethrough;
a second retaining plug having a shank extending from a head, wherein the shank is dimensioned to extend through the second mask connection opening and the second lens connection opening when the lens retaining assembly is connected with the lens and mask body, wherein the head has a greater dimension than the second mask connection opening to prevent the head from passing therethrough;
a first locking strap including a first segment spaced apart from a second segment, wherein the first segment and the second segment are configured to be attached to the first retaining plug, wherein when the first segment and the second segment are connected to the first retaining plug, a portion of the lens is retained on the first retaining plug between the first segment and the second segment; and
a second locking strap including a second segment spaced apart from a second segment, wherein the first segment and the second segment are configured to be attached to the second retaining plug, wherein when the first segment and the second segment are connected to the second retaining plug, a portion of the lens is retained on the second retaining plug between the first segment and the second segment.

2. The protective face mask with lens retaining assembly of claim 1, wherein the shank of the first retaining plug is dimensioned to extend longitudinally through the first mask connection opening and the first lens connection opening when the lens retaining assembly is connected with the lens and mask body.

3. The protective face mask with lens retaining assembly of claim 2, wherein the shank of the second retaining plug is dimensioned to extend longitudinally through the second mask connection opening and the second lens connection opening when the lens retaining assembly is connected with the lens and mask body.

4. The protective face mask with lens retaining assembly of claim 1, wherein at least one of the first locking strap and the second locking strap are flexible.

5. The protective face mask with lens retaining assembly of claim 1, wherein when the first locking strap is connected to the first retaining plug, the first segment and the second segment are separated by a portion of the lens.

6. The protective face mask with lens retaining assembly of claim 5, wherein when the second locking strap is connected to the second retaining plug, the first segment and the second segment are separated by a portion of the lens.

7. The protective face mask with lens retaining assembly of claim 1, wherein the first segment of the first locking strap has a hole dimensioned to receive at least a portion of the shank of the first retaining plug.

8. The protective face mask with lens retaining assembly of claim 7, wherein the first segment of the second locking strap has a hole dimensioned to receive the second retaining plug.

9. The protective face mask with lens retaining assembly of claim 7, wherein at least a portion of the first locking strap is resilient.

10. The protective face mask with lens retaining assembly of claim 9, wherein the hole in the first segment of the first locking strap is configured to deform to receive the shank of the first retaining plug.

11. The protective face mask with lens retaining assembly of claim 10, wherein the first retaining plug includes a retaining ring with an annular wall that has a greater dimension than the hole in the first segment of the first locking strap so that the hole in the first segment of the first locking strap must stretch to pass over the retaining ring.

12. The protective face mask with lens retaining assembly of claim 11, wherein the annular wall of the retaining ring includes an tapered edge.

13. The protective face mask with lens retaining assembly of claim 12, wherein the shank of the first retaining plug includes a radially extending fin.

14. The protective face mask with lens retaining assembly of claim 13, wherein the first segment of the first locking strap includes a notch in a perimeter of the hole configured to receive the fin in the first retaining plug.

15. The protective face mask with lens retaining assembly of claim 7, wherein the first retaining plug includes a passageway dimensioned to receive at least a portion of the second segment of the first locking strap.

16. The protective face mask with lens retaining assembly of claim 15, wherein the passageway extends along a transverse axis.

17. The protective face mask with lens retaining assembly of claim 16, wherein the second segment is configured to be received in the passageway.

18. The protective face mask with lens retaining assembly of claim 16, wherein the second segment includes one or more detents dimensioned to be received by one or more notches defined in the passageway when the second segment is received in the passageway.

19. The protective face mask with lens retaining assembly of claim 1, wherein the first retaining plug includes a first passageway dimensioned to receive the first segment of the first locking strap and a second passageway dimensioned to receive the second segment of the first locking strap, wherein the a portion of the lens is retained between the first segment and the second segment.

20. A protective face mask with lens retaining assembly comprising:
a mask body including a lens opening for receiving a lens, wherein the mask body defines a first mask connection opening and a second mask connection opening;
a lens dimensioned to be received in the lens opening, wherein the lens includes a first lens connection opening and a second lens connection opening alignable with the first mask connection opening and the second mask connection opening, respectively, of the mask body;
a lens retaining assembly comprising:
a first retaining plug having a shank extending from a head, wherein the shank is dimensioned to extend through the first mask connection opening and the first lens connection opening when the lens retaining assembly is connected with the lens and mask body, wherein the head has a greater dimension than the first mask connection opening to prevent the head from passing therethrough;
a second retaining plug having a shank extending from a head, wherein the shank is dimensioned to extend through the second mask connection opening and the second lens connection opening when the lens retaining assembly is connected with the lens and mask body, wherein the head has a greater dimension than the second mask connection opening to prevent the head from passing therethrough;
first means for retaining at least a portion of the lens on the first retaining plug, wherein the first means is flexed about its longitudinal axis; and
second means for retaining at least a portion of the lens on the second retaining plug, wherein the second means is flexed about its longitudinal axis.

21. A protective face mask with lens retaining assembly comprising:
a mask body including an opening for receiving a lens, wherein the mask body defines a first mask connection opening and a second mask connection opening;
a lens dimensioned to be received in the opening in the mask body, wherein the lens includes a first lens connection opening and a second lens connection opening alignable with the first mask connection opening and the second mask connection opening, respectively, of the mask body;
a lens retaining assembly comprising:
a first retaining plug dimensioned to extend through the first mask connection opening and the first lens connection opening;
a second retaining plug dimensioned to extend through the second mask connection opening and the second lens connection opening;
a first locking strap including a first segment spaced apart from a second segment, wherein the first segment is configured to be connected to the first retaining plug, wherein the second segment is moveable between a closed position connected to the first retaining plug such that the first locking strap captures at least a portion of the lens and an open position disconnected from the first retaining plug that releases the portion of the lens, wherein the first locking strap is flexed about its longitudinal axis when the second segment is connected to the first retaining plug; and
a second locking strap including a first segment spaced apart from a second segment, wherein the first segment is configured to be connected to the second retaining plug, wherein the second segment is moveable between a closed position connected to the second retaining plug such that the second locking strap captures at least a portion of the lens and an open position disconnected from the second retaining plug that releases the portion of the lens, wherein the second locking strap is flexed about its longitudinal axis when the second segment is connected to the second retaining plug.

22. The protective face mask with lens retaining assembly of claim 21, wherein the shank of the first retaining plug is dimensioned to extend longitudinally through the first mask connection opening and the first lens connection opening when the lens retaining assembly is connected with the lens and mask body.

23. The protective face mask with lens retaining assembly of claim 22, wherein the shank of the second retaining plug is dimensioned to extend longitudinally through the second mask connection opening and the second lens connection opening when the lens retaining assembly is connected with the lens and mask body.

24. The protective face mask with lens retaining assembly of claim 21, wherein at least one of the first locking strap and the second locking strap are formed from a resilient material.

25. The protective face mask with lens retaining assembly of claim 21, wherein when the first locking strap is connected to the first retaining plug, the first segment and the second segment are separated by a portion of the lens.

26. The protective face mask with lens retaining assembly of claim 25, wherein when the second locking strap is connected to the second retaining plug, the first segment and the second segment are separated by a portion of the lens.

27. The protective face mask with lens retaining assembly of claim 21, wherein the first segment of the first locking strap has a hole dimensioned to receive at least a portion of the shank of the first retaining plug.

28. The protective face mask with lens retaining assembly of claim 27, wherein the first segment of the second locking strap has a hole dimensioned to receive the second retaining plug.

29. The protective face mask with lens retaining assembly of claim 27, wherein at least a portion of the first locking strap is resilient.

30. The protective face mask with lens retaining assembly of claim 29, wherein the hole in the first segment of the first locking strap is configured to deform to receive the shank of the first retaining plug.

31. The protective face mask with lens retaining assembly of claim 30, wherein the first retaining plug includes a retaining ring with an annular wall that has a greater dimension than the hole in the first segment of the first locking strap so that the hole in the first segment of the first locking strap must stretch to pass over the retaining ring.

32. The protective face mask with lens retaining assembly of claim 31, wherein the annular wall of the retaining ring includes an tapered edge.

33. The protective face mask with lens retaining assembly of claim 32, wherein the shank of the first retaining plug includes a radially extending fin.

34. The protective face mask with lens retaining assembly of claim 33, wherein the first segment of the first locking strap includes a notch in a perimeter of the hole configured to receive the fin in the first retaining plug.

35. The protective face mask with lens retaining assembly of claim 37, wherein the first retaining plug includes a passageway dimensioned to receive at least a portion of the second segment of the first locking strap.

36. The protective face mask with lens retaining assembly of claim 35, wherein the passageway extends along a transverse axis.

37. The protective face mask with lens retaining assembly of claim 36, wherein the second segment is configured to be received in the passageway.

38. The protective face mask with lens retaining assembly of claim 36, wherein the second segment includes one or more detents dimensioned to be received by one or more notches defined in the passageway when the second segment is received in the passageway.

39. The protective face mask with lens retaining assembly of claim 31, wherein the first retaining plug includes a first passageway dimensioned to receive the first segment of the first locking strap and a second passageway dimensioned to receive the second segment of the first locking strap, wherein the a portion of the lens is retained between the first segment and the second segment.

40. A protective face mask with lens retaining assembly comprising:
a mask body including a lens opening for receiving a lens, wherein the mask body defines a first mask connection opening and a second mask connection opening;
a lens dimensioned to be received in the lens opening, wherein the lens includes a first lens connection opening and a second lens connection opening alignable with the first mask connection opening and the second mask connection opening, respectively, of the mask body;
a lens retaining assembly comprising:
a first retaining plug configured to extend longitudinally through the first mask connection opening and the first lens connection opening;
a second retaining plug configured to extend longitudinally through the second mask connection opening and the second lens connection opening;
a first locking strap configured to have at least two connection points with the first retaining plug, wherein at least a portion of the lens is captured between the connection points when the first locking strap is attached at the connection points with the first retaining plug; and
a second locking strap configured to have at least two connection points with the second retaining plug, wherein at least a portion of the lens is captured between the connection points when the second locking strap is attached at the connection points with the second retaining plug.

41. The protective face mask with lens retaining assembly of claim 40, wherein at least one connection point between the first locking strap and the first retaining plug is formed by a portion of the first locking strap being received within a passageway in the first retaining plug.

42. The protective face mask with lens retaining assembly of claim 41, the portion of the first locking strap received within a passageway in the first retaining plug includes a detent that is received by a notch within the passageway.

43. The protective face mask with lens retaining assembly of claim 41, wherein at least one connection point between the first locking strap and the first retaining plug is formed by a hole in the first locking strap through which at least a portion of the first retaining plug passes therethrough.

44. The protective face mask with lens retaining assembly of claim 43, wherein the hole in the first locking strap is configured to deform to receive the first retaining plug.

45. The protective face mask with lens retaining assembly of claim 44, wherein the first retaining plug includes a retaining ring with an annular wall that has a greater dimension than the hole in the first locking strap so that the hole in the first locking strap must stretch to pass over the retaining ring.

46. The protective face mask with lens retaining assembly of claim 45, wherein the annular wall of the retaining ring includes an tapered edge.

47. The protective face mask with lens retaining assembly of claim 46, wherein the first retaining plug includes a radially extending fin.

48. The protective face mask with lens retaining assembly of claim 47, wherein the first locking strap includes a notch in a perimeter of the hole configured to receive the fin in the first retaining plug.

49. The protective face mask with lens retaining assembly of claim 41, wherein the first locking strap flexes between the two connection points.

50. A protective face mask with lens retaining assembly comprising:
a mask body including a lens opening for receiving a lens, wherein the mask body defines a first mask connection opening and a second mask connection opening;
a lens dimensioned to be received in the lens opening, wherein the lens includes a first lens connection opening and a second lens connection alignable with the first mask connection opening and the second mask connection opening, respectively, of the mask body;
a lens retaining assembly comprising:
a first retaining plug having a shank extending from a head, wherein the shank is dimensioned to extend through the first mask connection opening and the first lens connection opening when the lens retaining assembly is connected with the lens and mask body, wherein the head has a greater dimension than the first mask connection opening to prevent the head from passing therethrough;
a second retaining plug having a shank extending from a head, wherein the shank is dimensioned to extend through the second mask connection opening and the second lens connection opening when the lens retaining assembly is connected with the lens and mask body, wherein the head has a greater dimension than the second mask connection opening to prevent the head from passing therethrough;
a first locking strap including a first segment spaced apart from a second segment, wherein the first segment has a hole dimensioned to receive at least a portion of the shank of the first retaining plug, wherein the hole in the first segment of the first locking strap is configured to resiliently deform to receive the shank of the first retaining plug, wherein the second segment is configured to be received by a passageway defined in the first retaining plug, wherein the second segment includes one or more detents dimensioned to be received by one or more notches defined in the passageway when the second segment is received in the passageway, wherein when the first segment and the second segment are connected to the first retaining plug, a portion of the lens is retained on the first retaining plug between the first segment and the second segment; and
a second locking strap including a first segment spaced apart from a second segment, wherein the first segment has a hole dimensioned to receive at least a portion of the shank of the second retaining plug, wherein the hole in the first segment of the second locking strap is configured to resiliently deform to receive the shank of the second retaining plug, wherein the second segment is configured to be received by a passageway defined in the second retaining plug, wherein the second segment includes one or more detents dimensioned to be received by one or more notches defined in the passageway when the second segment is received in the passageway, wherein when the first segment and the second segment are connected to the second retaining plug, a portion of the lens is retained on the second retaining plug between the first segment and the second segment.

51. A method for connecting a lens to a mask, the method comprising the steps of:
providing a mask body including a lens opening for receiving a lens, wherein the mask body defines at least one mask connection opening;
providing a lens dimensioned to be received in the lens opening, wherein the lens defines at least one lens connection opening;
aligning the mask connection opening with the lens connection opening;
inserting a retaining plug through the mask connection opening and the lens connection opening;
connecting a first portion of a locking strap to the retaining plug on a first side of the lens;
connecting a second portion of the locking strap to the retaining plug on a second side of the lens; and
wherein at least a portion of the lens is retained on the retaining plug between the first portion and the second portion of the locking strap.

52. The method of claim 51, wherein the first portion of the locking strap includes a hole, wherein connecting the first portion of the locking strap to the retaining plug includes the step of inserting the retaining plug through the hole.

53. The method of claim 52, wherein the retaining plug stretches the hole in the first portion of the locking strap when the first portion is connected to the retaining plug.

54. The method of claim 51, wherein the locking strap flexes when the second portion of the locking strap is connected to the retaining plug.

55. The method of claim 51, wherein the retaining plug includes a passageway dimensioned to receive the second portion of the locking strap, wherein connecting the second portion of the locking strap to the retaining plug includes the step of inserting the second portion of the locking strap into the passageway in the retaining plug.
